(19) United States
(12) Patent Application Publication
(10) Pub. No.: US 2025/0041211 A1
(43) Pub. Date: Feb. 6, 2025

(54) OPHTHALMIC PREPARATION AND APPLICATION THEREOF IN TREATMENT OF PRESBYOPIA

(71) Applicant: EYE HOSPITAL OF WENZHOU MEDICAL UNIVERSITY, Wenzhou (CN)

(72) Inventor: Wei Chen, Wenzhou (CN)

(73) Assignee: EYE HOSPITAL, WENZHOU MEDICAL UNIVERSITY, Wenzhou (CN)

(21) Appl. No.: 18/922,397

(22) Filed: Oct. 22, 2024

Related U.S. Application Data
(63) Continuation of application No. PCT/CN2023/089795, filed on Apr. 21, 2023.

(30) Foreign Application Priority Data
Apr. 22, 2022 (CN) CN2022104320535

(51) Int. Cl. A61K 9/00; A61P 27/10

(52) U.S. Cl. CPC A61K 9/0048; A61P 27/10

(57) ABSTRACT

The present invention discloses an ophthalmic preparation and an application thereof in the treatment of presbyopia. The ophthalmic preparation includes aceclidine and rebamipide. It is found and proved in the present invention for the first time that the aceclidine and the rebamipide are combined to produce a synergistic effect, the rebamipide can enhance an effect of the aceclidine and reduce related side effects, and the aceclidine can effectively produce a synergistic effect with the rebamipide to contract the pupillary sphincter and have a dosage effect. The ophthalmic preparation can effectively improve, alleviate or treat presbyopia, have a potential effect of slowing down the progression of the presbyopia course, and have a very good clinical application prospect.

### CROSS-REFERENCE TO RELATED APPLICATION

The present application claims the priority of the invention patent application filed on Apr. 22, 2022 with the application number of CN2022104320535 and the title of “Ophthalmic Preparation and Application Thereof in the Treatment of Presbyopia”, which is incorporated herein in its entirety by reference.

### TECHNICAL FIELD

The present application belongs to the technical field of biological medicine, and more particularly, relates to an ophthalmic preparation and an application thereof in the treatment of presbyopia. In particular, the ophthalmic preparation in the present application includes aceclidine and rebamipide.

### BACKGROUND ART

As people age, their eye accommodation ability gradually declines, leading to difficulty in seeing close objects. This phenomenon is called presbyopia, also known as Pb. For a long time, the main methods used to correct presbyopia were reading glasses or bifocal glasses, and special contact lenses designed for presbyopia, as well as several surgical treatments, including the implantation of artificial lenses, corneal laser correction, etc. Currently, the most widely used method is optical glasses for correction. Such glasses are suitable for temporary wear during reading, but can only be used to see objects at a close range during wear, not objects at a medium distance, so the power of presbyopic glasses during optometry needs to be adjusted and reduced in the event of seeing objects at a medium distance. In daily life and work, people with myopia or hyperopia must have two pairs of glasses alternately, which is very inconvenient. In addition, this correction scheme cannot cure or alleviate presbyopia pathologically. Therefore, it is of great significance to develop a drug-related treatment method that avoids the use of similar devices. According to statistics, there are about 1.1 billion presbyopic patients in the world, with an incidence of 35.6% in people aged 35 and above, and 40.3% in people aged 40 and above.

The changes in the position and shape of the lens resulting from the contraction of the ciliary muscle of the eye are the main mechanism of human eye focusing. However, from childhood, the lens gradually loses its malleability and its strain ability to the contraction of the ciliary muscle. As we age, after the age of 40, due to the continuous loss of malleability in the lens and a decline in the strain ability to the contraction of the ciliary muscle, the eyes cannot focus on close objects in a comfortable way, resulting in the development of presbyopia. There are two main mechanisms for the treatment of presbyopia: firstly; the ciliary muscle contraction is regulated. The ciliary muscle is controlled by acetylcholine and a muscarinic receptor under the control of a parasympathetic nervous system. Secondly, the pupil diameter is changed, and the diastolic muscle and sphincter muscle of the iris are regulated. The sphincter muscle of the iris is mainly controlled by the parasympathetic nervous system of the muscarinic receptor (M-type receptor), while the diastolic muscle is mainly controlled by a sympathetic nervous system. Therefore, activating the sphincter muscle of the iris or relaxing the diastolic muscle of the iris of a presbyopic patient with appropriate stimulation can achieve an effect of pupil contraction, thereby increasing the depth of the eye's visual field and alleviating presbyopia.

Previous treatments for presbyopia have tried various combinations of drugs, such as pilocarpine and a non-steroidal anti-inflammatory agent, pilocarpine and brimonidine, pilocarpine and dapiprazole, etc. Long-term use of certain prescription drugs may cause serious ocular side effects. For example, the use of diclofenac is significantly associated with corneal dissolution and perforation (US Patent 2010/0016395), and the use of pilocarpine at a concentration above 0.5% (w/v) is associated with side effects such as red eye, eye pain, and migraine, limiting the clinical use of such drugs. Meanwhile, due to the continuous course of presbyopia, there is currently no drug that has the potential to slow down the long-term course of presbyopia while ensuring a pupil contraction duration. The only drug currently with the potential to alleviate the course is choline lipoate (UNR844-Cl), which reduces disulfide bonds in the lens. Over time, the disulfide bonds limit the shape change of the lens through contraction and relaxation of the ciliary muscle. Age-related disulfide bonds also contribute to the development of nuclear sclerosing cataracts. Rebamipide (Reb) was first used as a gastrointestinal protective drug for the treatment of gastric ulcers and gastritis, as well as the improvement of inflammations by increasing blood flow to the gastric mucosa, prostaglandin E2 synthesis and gastric mucus secretion, scavenging oxygen free radicals, etc. Recent studies have shown that rebamipide can be used for the treatment of dry eye, and eye drops were officially used in Japan in 2012 for the treatment of dry eye diseases, with good clinical safety and no long-term side effects. Rebamipide eye drops can effectively reduce the release of inflammatory cytokines, have an effect of promoting mucin production and secretion, and also have the potential to alleviate the progression of the long-term course of aging-related diseases due to its strong antioxidant effect.

At present, there is still an urgent need in the field for a non-invasive, user-friendly drug treatment method with no or minimal side effects. Specifically, the efficacy should be achieved so that both eyes of the presbyopic patients can focus on close objects, with a duration of use that should be longer than the daily eye use time (six hours) and without any risks of decreased distant vision, eye irritation (pain, redness), retinal detachment, or corneal perforation, etc. To date, there have been no reports of using aceclidine in combination with rebamipide for the treatment of presbyopia.

### SUMMARY OF THE INVENTION

In view of this, in order to overcome the above-mentioned technical problems existing in the art at present, an object of the present application is to provide an ophthalmic preparation and an application thereof in the treatment of presbyopia. Meanwhile, the inventors of the present application are surprised to find that the efficacy of the ophthalmic preparation is enhanced by combined use of rebamipide and aceclidine, which have a synergistic effect.

The above object of the present application is achieved by the following technical solutions.

A first aspect of the present application provides an ophthalmic preparation for improving, alleviating or treating the presbyopia.

Further, the ophthalmic preparation contains effective amounts of muscarinic acetylcholine receptor M3 agonist and rebamipide.

Further, the muscarinic acetylcholine receptor M3 agonist includes aceclidine, pilocarpine, carbachol, methacholine, cevimeline, talsaclidine, acetylcholine, huperzine A, milameline, DREADD agonist 21, alvameline, carbamylmethylcholine, sabcomeline, and/or arecoline.

Further, the muscarinic acetylcholine receptor M3 agonist is aceclidine.

Further, the ophthalmic preparation further contains an α-2 adrenergic receptor agonist.

Further, the α-2 adrenergic receptor agonist includes brimonidine, medetomidine, guanfacine, clonidine, dexmedetomidine, apraclonidine, mivazerol, naphazoline, oxymetazoline, tetrahydrozoline, xylazine, tizanidine, methylnorepinephrine, moxonidine, and/or rilmenidine.

Further, the ophthalmic preparation also contains a viscosity enhancer.

Further, the viscosity enhancer includes carboxymethyl cellulose, methylcellulose, hydroxypropyl methylcellulose, polyethylene glycol, povidone, glycerin, polyvinyl alcohol, polyvinylpyrrolidone, polyalkyl styrene, polymethacrylate, and/or polyacrylate.

Further, the viscosity enhancer is carboxymethyl cellulose.

Further, the ophthalmic preparation further contains a surfactant.

Further, the surfactant includes hydroxypropyl-β-cyclodextrin, polyoxyethylene alkyl ether, Tween, sodium lauryl sulfate, sodium lauryl sulfovinate, poloxamer, polysorbate, sorbitan monopalmitate, sorbitan monostearate, sorbitan monooleate, and/or polyethylene glycol alkyl.

Further, the surfactant is hydroxypropyl-β-cyclodextrin.

Further, the surfactant is not limited to the surfactants listed in the present application, and any anionic surfactant, nonionic surfactant or a combination thereof that may be added to a pharmaceutical product is within the protection scope of the present application.

Further, the anionic surfactant includes (but is not limited to): γ-cyclodextrin, sulfobutyl ether β-cyclodextrin, sodium lauryl sulfate and sodium laureth sulfate. The nonionic surfactant includes (but is not limited to): poloxamer, tyloxapol, polysorbate, sorbitan monolaurate, sorbitan monopalmitate, sorbitan monostearate, sorbitan monooleate, polyethylene glycol stearate, polyethylene glycol alkyl, cyclodextrin and derivatives thereof.

Further, the aceclidine has a concentration of 0.50%-1.50% (w/v).

Further, the rebamipide has a concentration of 0.50%-4.00% (w/v).

Further, the brimonidine has a concentration of 0.01%-1.00% (w/v).

Further, the carboxymethyl cellulose has a concentration of 0.03%-0.50% (w/v).

Further, the hydroxypropyl-β-cyclodextrin has a concentration of 0.01%-5.00% (w/v).

In some embodiments, the ophthalmic preparation provided in the present application for improving, alleviating, or treating the presbyopia includes aceclidine (also known as: quinuclidin-3-yl acetate, 1-azabicyclo[2.2.2]oct-3-yl acetate, aceclidine, (3R)-1-azabicyclo[2.2.2]oct-3-yl acetate, 1-azabicyclo[2.2.2]-3-octyl acetate) or pilocarpine (also known as: 4-[(1-methyl-1H-imidazol-5-yl)methyl]-3-ethyldihydro-2(3H)-furanone), and a muscarinic agonist of a pharmaceutically acceptable salt, ester, analogue or derivative thereof; and rebamipide (also known as: 4-[(4-chlorobenzoyl)amino]-1,2-dihydro-2-oxo-4-quinoline propionic acid, a-[(4-chlorobenzoyl)amino]-1,Chemicalbook2-dihydro-2-oxo-4-quinoline propionic acid, 2-(4-chlorobenzamido)-3-(1,2-dihydro-2-oxo-4-quinolineyl) propionic acid) and a pharmaceutically acceptable salt, ester, analogue, prodrug or derivative thereof.

In optional embodiments, the ophthalmic preparation provided in the present application for improving, alleviating or treating the presbyopia includes aceclidine and rebamipide.

In an optional embodiment, the ophthalmic preparation provided in the present application for improving, alleviating or treating the presbyopia includes aceclidine, rebamipide and an α-2 adrenergic receptor agonist, wherein the α-2 adrenergic receptor agonist includes (but is not limited to): brimonidine, medetomidine, guanfacine, clonidine, dexmnedetomidine, apraclonidine, mivazerol, naphazoline, oxymetazoline, tetrahydrozoline, xylazine, tizanidine, methylnorepinephrine, moxonidine, and rilmenidine.

In another optional embodiment, the ophthalmic preparation provided in the present application for improving, alleviating or treating the presbyopia include aceclidine, rebamipide, and brimonidine.

In another optional embodiment, the ophthalmic preparation provided in the present application for improving, alleviating or treating the presbyopia include aceclidine, rebamipide, brimonidine, a viscosity enhancer and a surfactant, wherein the viscosity enhancer includes (but is not limited to): carboxymethyl cellulose, methylcellulose, hydroxypropyl methylcellulose, polyethylene glycol, povidone, glycerin, polyvinyl alcohol, polyvinylpyrrolidone, polyalkyl styrene, polymethacrylate, and polyacrylate; and the surfactant includes (but is not limited to): hydroxypropyl-β-cyclodextrin, polyoxyethylene alkyl ether, Tween, sodium lauryl sulfate, sodium lauryl sulfovinate, poloxamer, polysorbate, sorbitan monopalmitate, sorbitan monostearate, sorbitan monooleate, and polyethylene glycol alkyl.

In another optional embodiment, the ophthalmic preparation provided in the present application for improving, alleviating or treating the presbyopia includes aceclidine, rebamipide, brimonidine, carboxymethyl cellulose and hydroxypropyl-β-cyclodextrin.

In another optional embodiment, the ophthalmic preparation provided in the present application for improving, alleviating or treating the presbyopia includes aceclidine, rebamipide, brimonidine, carboxymethyl cellulose, hydroxypropyl-β-cyclodextrin, sodium chloride, benzalkonium chloride, and sodium hydroxide.

Further, the sodium chloride is a tension regulator, and may also be replaced with potassium chloride, mannitol or glycerin, or other pharmaceutically or ophthalmologically acceptable tension regulators.

Further, the benzalkonium chloride is a preservative, and may also be replaced with sorbic acid, oxychlorine complex, citric acid, chlorobutanol, thiomersal, phenylmercuric acetate, disodium ethylenediamine tetraacetic acid, phenylmercuric nitrate, perborate or benzyl alcohol.

Further, the sodium hydroxide is a pH regulator, and may also be replaced with an acetate buffer, a citrate buffer, a phosphate buffer or a borate buffer.

In another optional embodiment, the ophthalmic preparation provided in the present application for improving, alleviating or treating the presbyopia includes:
aceclidine, having a concentration of about 0.50% to about 1.50% (w/v);
rebamipide, having a concentration of about 0.50% to about 4.00% (w/v);
brimonidine, having a concentration of about 0.01% to about 1.00% (w/v);
carboxymethyl cellulose, having a concentration of about 0.03% to about 0.50% (w/v);
hydroxypropyl-β-cyclodextrin, having a concentration of about 0.01% to about 5.00% (w/v);
sodium chloride, having a concentration of about 0.01% to about 2.00% (w/v);
benzalkonium chloride, having a concentration of about 0.001% to about 0.50% (w/v); and
sodium hydroxide, having a concentration of about 1-5 mmol, and a pH being adjusted to 6.9.

Further, the ophthalmic preparation may also include pharmaceutically acceptable carriers and/or accessories.

Further, each pharmaceutically acceptable carrier and/or accessory means a material, composition or excipient, such as a liquid or solid filler, a diluent, a solvent or a coating material, that is recognized in the art and includes, for example, those involved in carrying or transporting any subject composition from one organ or part of the body to another organ or part of the body. Each carrier must be “acceptable” in the sense of compatibility with other components of the subject composition and harmless to the patient. In some embodiments, the pharmaceutically acceptable carrier and/or accessory is pyrogen-free. Some examples of materials that may be used as pharmaceutically acceptable carriers and/or accessories include: (1) sugars, such as lactose, glucose, and sucrose; (2) starches, such as corn starch and potato starch; (3) cellulose and derivatives thereof, such as sodium carboxymethylcellulose, ethylcellulose, and cellulose acetate; (4) powdered tragacanth gum; (5) malt; (6) gelatin; (7) talcum powder; (8) cocoa butter and suppository wax; (9) oils, such as peanut oil, cottonseed oil, sunflower oil, sesame oil, olive oil, corn oil, and soybean oil; (10) diols, such as propylene glycol; (11) polyols, such as glycerin, sorbitol, mannitol and polyethylene glycol; (12) esters, such as ethyl oleate and ethyl laurate; (13) agar; (14) buffers, such as magnesium hydroxide and aluminum hydroxide; (15) alginic acid; (16) pyrogen-free water; (17) isotonic saline; (18) Ringer's solution; (19) ethanol; (20) phosphate buffer; and (21) other non-toxic compatible substances used in pharmaceutical preparations. The pharmaceutically acceptable carriers and/or accessories described in the present application are not limited to the substances listed in the present application, and all carriers and/or accessories that can be used in ophthalmic preparations should fall within the protection scope of the present application.

In a specific embodiment of the present application, the present application proves through experiments that the combination of aceclidine and rebamipide in the ophthalmic preparation has a synergistic therapeutic effect on the presbyopia, and also has the potential of slowing down the long-term course of the presbyopia under the condition of ensuring the duration of pupil contraction. In addition, the co-administration of aceclidine and rebamipide can be used for the following aspects: 1) the treatment of a variety of eye diseases, including presbyopia, mild hyperopia, irregular astigmatism, or hyperopic accommodative esotropia; 2) the increase in the visual depth of field; 3) pupil contraction, which may reach about 1.2 to about 2 mm, with the duration of pupil contraction being at least about 6 hours; and 4) alleviation of the progression of presbyopia.

In some embodiments, the ophthalmic preparation provided in the present application may be used alone or in combination with other therapeutic agents used to improve, alleviate, or treat the presbyopia.

Further, the other therapeutic agents include a miotic.

Further, the miotic includes an α-1 adrenergic receptor antagonist, a D-adrenergic receptor antagonist, a nicotinic receptor agonist, an antipsychotic drug, an antiemetic, cannabinoid, a MAO inhibitor, an EP1 receptor agonist, an EP4 receptor agonist, an FP receptor agonist, and a calcium channel modulator.

Further, the α-1 adrenergic receptor antagonist includes phenoxybenzamine, phentolamine, tolazoline, trazodone, alfuzosin, dapiprazole, thymoxamine, doxazosin, prazosin, tamsulosin, bunezosin, terazosin, trimazosin, silodosin, atipamezole, idazoxan, mirtazapine, yohimbin, carvedilol, labetalol, urapidil, abanoquil, adimolol, ajmalicine, amosulalol, arotinolol, atiprosin, benoxathian, buflomedil, bunazosin, carvedilol, CI-926, corynanthine, DL-017, domesticine, eugenodilol, fenspiride, GYKI-12743, GYKI-16084, indoramine, ketanserin, L-765314, mephendioxan, Metazosin, monatepil, naftopidil, nantenine, beldazosin, nicergoline, niguldipine, pelanserin, phendioxan, piperoxan, quinazosin, ritanserin, RS-97078, SGB-1534, SL-890591, spiperone, talipexole, tibalosin, tiodazosin, tipentosin, tolazoline, upidosin, zolertine, or a pharmaceutically acceptable salt of any one or more of the above compounds, or combinations thereof.

Further, the β-adrenergic receptor antagonist includes acebutolol, atenolol, betaxolol, bisoprolol, carteolol, esmolol, isoprenaline, levobunolol, metoprolol, penbutolol, nadolol, nebivolol, indolol, propranolol, timolol, sotalol, etc., and/or a pharmaceutically acceptable salt of any one or more of the above compounds, or combinations thereof.

Further, the nicotinic receptor agonist includes nicotine, varenicline, galantamine, epibatidine, lobeline, decamethonium, cytosine, nifene, dimethylphenylpiperazinium, etc., and/or a pharmaceutically acceptable salt of any one or more of the above compounds, or combinations thereof.

Further, the antipsychotic drug includes risperdal, haloperidol, chlorpromazine, olanzapine, quetiapine, mirtazapine, chlorpromazine, prochlorperazine, alizapride, metoclopram, midazolam, lorazepam, etc., and/or a pharmaceutically acceptable salt of any one or more of the above compounds, or combinations thereof.

Further, the antiemetic includes ondansetron, droperidol, metoclopram, dolasetron, granisetron, tropisetron, palonosetron, domperidone, aprepitant, casopitant, rolapitant, cyclizene, diphenhydramine, dimenhydrinate, doxylamine, meclizine, promethazine, hydroxyzine, etc., and/or a pharmaceutically acceptable salt of any one or more of the above compounds, or combinations thereof.

Further, the cannabinoid includes cannabis, dronebinol, nabilone, sativex, etc., and/or a pharmaceutically acceptable salt of any one or more of the above compounds, or combinations thereof.

Further, the MAO (monoamine oxidase) inhibitor includes selegiline, befloxatone, moclobemide, isocarboxazid, nialamide, phenelzine, hydracarbazine, tranylcypromine, bifemelane, pirlindole, toloxatone, rasagiline, linezolid, methylene blue, etc., and/or a pharmaceutically acceptable salt of any one or more of the above compounds, or combinations thereof.

Further, the EP1 receptor agonist, the EP4 receptor agonist and the FP receptor agonist include PGE2, PGE1, PGF2α, PGD2, PGE2, PGI2, TXA2, cloprostenol, fluprostenol, latanoprost, tafluprost, enprostil, sulprostone, U46619, carbacyclin and iloprost, ONO-D1-004, 1-hydroxy-PGE1, rivenprost (ONO-4819), OOG-308, ONO-AE1-329, AGN205203, ONO-4819, CP-734432, AE1-329, SC-19220, SC-51089, EP4RAG, etc., and/or a pharmaceutically acceptable salt of any one or more of the above compounds, or combinations thereof.

Further, the other therapeutic agents include antibiotics, steroids, artificial tears, intraocular pressure (IOP) lowering agents, immunosuppressants, therapeutic agents for dry eyes, etc.

Further, the ophthalmic preparation provided in the present application may be used concurrently, separately or sequentially when used in combination with other therapeutic agents used to improve, alleviate or treat the presbyopia.

In some embodiments, the dosage forms of the ophthalmic preparation provided in the present application include (but are not limited to): any pharmaceutical dosage form, such as eye drops, ophthalmic ointments, ophthalmic gels, injections, oral immediate-release agents, and implants. In a specific embodiment, the ophthalmic preparation provided in the present application may be prepared into various dosage forms according to actual needs, and applied by the clinicians according to the type, age, weight and general disease status of a subject, an administration mode and other factors that are beneficial to the subject. The administration mode may be any appropriate administration mode known to those skilled in the art.

Further, the present application provides a kit. The kit includes the ophthalmic preparation according to the first aspect of the present application and a manual for applying the ophthalmic preparation to the eyes of a subject in need. In one embodiment, the ophthalmic preparation is provided or packaged in a plurality of dosage forms. In another embodiment, the ophthalmic preparation contains a preservative that prevents microbial contamination during use (i.e., repeated use). The application manual provides an administration description. In various embodiments, the ophthalmic preparation may be applied once a day, twice a day, or three times a day in the description. In an embodiment where the ophthalmic preparation is a liquid preparation, the ophthalmic preparation may be applied with one, two, three or more drops to one or both eyes (e.g., if one eye or both eyes is or are affected by an ocular symptom, both eyes can be treated), once a day, twice a day, three or more times a day.

A second aspect of the present application provides an application of a muscarinic acetylcholine receptor M3 agonist in combination with rebamipide in the preparation of an ophthalmic preparation for improving, alleviating or treating the presbyopia.

Optionally, the muscarinic acetylcholine receptor M3 agonist includes aceclidine, pilocarpine, carbachol, methacholine, cevimeline, talsaclidine, huperzine A, acetylcholine, milameline, DREADD agonist 21, alvameline, carbamylmethylcholine, sabcomeline, or arecoline.

Optionally, the muscarinic acetylcholine receptor M3 agonist is aceclidine.

Further, the ophthalmic preparation further contains an α-2 adrenergic receptor agonist.

Optionally, the α-2 adrenergic receptor agonist includes brimonidine, medetomidine, guanfacine, clonidine, dexinedetomidine, apraclonidine, mivazerol, naphazoline, oxymetazoline, tetrahydrozoline, xylazine, tizanidine, methylnorepinephrine, moxonidine, or rilmenidine.

Optionally, the α-2 adrenergic agonist is brimonidine.

Further, the aceclidine has a concentration of 0.50%-1.50% (w/v).

Optionally, the rebamipide has a concentration of 0.50%-4.00% (w/v).

Optionally, the brimonidine has a concentration of 0.01%-1.00% (w/v).

A third aspect of the present application provides an application of the ophthalmic preparation according to the first aspect of the present application in the preparation of a pharmaceutical composition for improving, alleviating or treating the presbyopia.

Further, the pharmaceutical composition further contains pharmaceutically acceptable carriers and/or accessories.

Further, the pharmaceutically acceptable carriers and/or accessories are well documented in Remington's Pharmaceutical Sciences (19th ed., 1995). These substances are used as needed to aid in the stability of the formulation or to help increase the activity or its bioavailability or, in the case of an eye drop experiment, to efficiently deliver the drug to posterior segments of the eyes. A preparation that can be used in this pharmaceutical composition may be in the form of its original compound itself or, optionally, in the form of its pharmaceutically acceptable salts. The pharmaceutical composition so formulated may be administrated in any appropriate manner known to those skilled in the art as needed.

Further, the appropriate administration dosage of the pharmaceutical composition may be prescribed in a variety of ways according to a preparation method, an administration mode, the patient's age, weight, sex, condition, diet, administration time, administration route, excretion rate and response sensitivity and other factors, and a skilled physician can often easily determine a prescription and an appropriate administration dosage for the desired treatment or prevention. A therapeutically effective amount and a specific treatment regimen for a certain object (such as mammal: human) can be affected by many factors, including: the pharmacodynamic activity of a drug used, the subject's age, weight, general condition, gender, diet, administration time, disease susceptibility and disease course, judgments from a treating physician, etc. In addition, a person skilled in the art knows that the administration mode, dosage form and administration dosage are affected by many factors such as the patient's age, weight, gender, condition, diet, administration time, excretion rate, and response sensitivity. Therefore, the administration mode, the dosage form, and the administration dosage of the ophthalmic preparation or pharmaceutical composition described in the present application are not limited to the administration modes, the dosage forms and the administration dosages described in the examples of the present application.

The present application further provides a method for preparing an ophthalmic preparation according to the first aspect of the present invention.

Further, the method includes: combining effective amounts of muscarinic acetylcholine receptor M3 agonist and rebamipide with a pharmaceutically acceptable carrier and/or accessory.

A fourth aspect of the present application provides a method for improving, alleviating or treating presbyopia, mild hyperopia, irregular astigmatism and/or hyperopic accommodative esotropia, or increasing the visual depth of field, or contracting pupils, or alleviating the progression of a long-term or medium-term course of presbyopia.

Further, the method includes: applying an effective amount of the ophthalmic preparation according to the first aspect of the present application to an affected eye of a subject in need.

Further, the subject refers to any animal, or human and non-human animals. The non-human animals include all vertebrates, e.g., mammals, such as non-human primates (especially higher primates), sheep, dogs, rodents (e.g., mice or rats), guinea pigs, goats, pigs, cats, rabbits, cattle, and any domestic animals or pets; and non-mammals, such as chicken, amphibians, and reptiles. In a specific embodiment of the present application, the subject is preferably human.

In some embodiments, the method for applying the effective amount of the ophthalmic preparation according to the first aspect of the present application to the affected eye of the subject in need includes topical application, subconjunctival application, intravitreal application, and systematic delivery.

Topical application: topical ocular drug application is usually accomplished with eye drops. The contact time on the surface of the eye is short, but may be extended with specific preparations (e.g., gels, gelling preparations, ointments, and inserts). The basic nature of a solution containing the pharmaceutical composition is generally aqueous, so reagents aimed at increasing the viscosity of the solution can be used. Such reagents include, for example, hydroxypropyl methylcellulose, carbomer, polyvinyl alcohol, etc.

Subconjunctival application: traditionally, subconjunctival injection has been used to deliver the drug to the uvea at increasing levels. This application mode may be used to deliver a drug in a controlled-release preparation to the posterior segment and to guide the healing process after surgery.

Intravitreal application: direct drug application to the vitreous body provides the advantage of more direct access to the vitreous body and retina. However, the delivery from the vitreous body to the choroid is more complex due to obstruction by a retinal pigment epithelium (RPE) barrier. Small molecules can diffuse rapidly in the vitreous body, but the migration of large molecules, especially those that are positively charged, is limited. An injectable composition suitable for intraocular injection usually contains a drug solution or a fine particle suspension that allows for continuous delivery to the eyes. The preparation is typically aqueous and may generally include solubilizers such as, but not limited to, polyvinyl alcohol, Tween 80, solutol, cremophore, and cyclodextrin. These solubilizers can be combined. The preparation is typically in a pH range of 3 to 8, which is considered acceptable for intravitreal preparations. To achieve an acceptable pH, buffer systems are sometimes used. These buffer systems include (but are not limited to) citrate and phosphate-based buffering systems. The tension of the intravitreal preparation may be adjusted to maintain a desired range typically of 250 to 360 mOsm/kg. The tension adjustment may be achieved, for example, by the addition of sodium chloride. Typically, an intravitreal preparation is produced by aseptic preparation for single use. Preservative preparations, e.g., those containing preservatives such as benzyl alcohol, may be used. A dosage of an active agent in the composition of the present application will depend on the nature and extent of the condition, the age and condition of the patient, and other factors known to those skilled in the art. The application may be given as a single injection without further administration, or as a plurality of injections.

System delivery: the delivery is performed using an ocular drug delivery system. The ocular drug delivery system includes (but is not limited to): ocular implants, anterior chamber implants, intravitreal implants, subconjunctival implants, sub-Tenon's implants, punctal plugs, tubule-eluting implants, and ocular rings.

In some embodiments, the ophthalmic preparation provided in the present application is applied to one or both eyes of a patient exhibiting symptoms of presbyopia to improve the patient's ability to focus on objects at a close distance, including objects around normal reading distances. In some embodiments, the application of the ophthalmic preparation provided in the present application does not depend on other therapeutic methods to adequately improve the patient's near vision acuity. For example, the administration of the ophthalmic preparation provided in the present application may enable the patient to focus on an object at a distance around the normal reading distance without the use of corrective lenses or corrective eye surgery. For patients in the early stages of presbyopia, the administration of the ophthalmic preparation provided in the present application can alleviate presbyopia-related symptoms, including making near vision acuity possible without the use of corrective lenses or glasses.

In some embodiments, the ophthalmic preparation provided in the present application is administered to patients with presbyopic symptoms (other than those from myopia or hyperopia) to facilitate near vision acuity, so that patients do not need to rely on corrective treatments such as bifocal/multifocal lenses or monocular contact lenses, or do not need to remove their glasses to read in myopic eyes.

In some embodiments, the ophthalmic preparation provided in the present application is administered to one or both eyes of the patient to provide treatment for presbyopia as an alternative to corrective eye surgery. For example, the ophthalmic preparation provided in the present application may be administered to a patient with presbyopic symptoms when the patient cannot undergo corrective eye surgery to treat presbyopia. The ophthalmic preparation provided in the present application may also be administered to myopic or hyperopic patients with presbyopia symptoms (with or without astigmatism), who preferably accept the treatment for distant vision defects only. Alternatively, the ophthalmic preparation provided in the present application may be administrated to a patient who continues to have presbyopic symptoms after the patient has already undergone corrective eye surgery for presbyopia. The ophthalmic preparation provided in the present application may be used in conjunction with corrective eye surgery for presbyopia to further alleviate presbyopic symptoms. In some embodiments, the ophthalmic preparation provided in the present application is administered to one or both eyes of a patient to reduce the decline in near vision acuity after corrective ocular surgery for distant vision at a younger age.

In some embodiments, the administration of the ophthalmic preparation provided in the present application is beneficial for the improvement of presbyopic symptoms in patients who have been reversed by a previous corrective eye surgery for presbyopia. For example, after a previous monocular laser surgery for presbyopia has been reversed or resolved, the administration of the ophthalmic preparation to the patient may allow for the reconstruction of binocular vision.

In some embodiments, the ophthalmic preparation provided in the present application is administrated to the patient to improve the patient's ability to focus on nearby objects after the patient has undergone corrective surgery for the treatment of an ocular condition other than presbyopia (e.g., including a corrective eye surgery for the treatment of cataracts).

In some embodiments, the ophthalmic preparation provided in the present application is used in conjunction with other treatments for ocular conditions, including those for presbyopic symptoms. For example, in conjunction with the use of a monofocal intraocular lens, a multifocal intraocular lens, or an accommodative intraocular lens, the ophthalmic preparation provided in the present application may be administrated to the patient to improve the patient's near-focusing ability.

A fifth aspect of the present application provides an application of the ophthalmic preparation according to the first aspect of the present application in any of the following aspects:
(1) the application of the ophthalmic preparation according to the first aspect of the present application in the treatment of presbyopia, mild hyperopia, irregular astigmatism and/or hyperopic accommodative esotropia;
(2) the application of the ophthalmic preparation according to the first aspect of the present application in the increase in the visual depth of field;
(3) the application of the ophthalmic preparation according to the first aspect of the present application in pupil contraction; and
(4) the application of the ophthalmic preparation according to the first aspect of the present application in alleviating the progression of the long-term and medium-term course of presbyopia.

Compared with the prior art, the present application has the following advantages and beneficial effects.

The present application discloses for the first time a composition of aceclidine and rebamipide for the treatment of eye disorders, including presbyopia, mild hyperopia, and irregular astigmatism. It is surprisingly found that the composition provided in the present application can enhance the effect of aceclidine and reduce related side effects, and after combined use, the aceclidine can effectively produce a synergistic effect with the rebamipide to contract the pupillary sphincter. In addition, the composition provided in the present application may also be used to delay or reverse the aging process of the lens and surrounding tissues, and delay the long-term progression of presbyopia, and is especially suitable for the treatment of patients with presbyopia and dry eye.

### BRIEF DESCRIPTION OF THE DRAWINGS

The embodiments of the present application will be described in detail below with reference to the accompanying drawings. In drawings:

FIG. 1 is a screening result diagram of drug combination based on target information, where Panel A shows screening in a combined mode of a screened drug with COX-2, and Panel B shows screening in a combined mode of a screened drug and a muscarinic acetylcholine receptor.

FIG. 2 is a morphology diagram of solutions corresponding to Prescription #3, Prescription #2, and Prescription #12 eye drops, where Panel A shows Prescription #3, Panel B shows Prescription #2, and Panel C shows Prescription #12;

FIG. 3 is a statistical chart of elasticity results of mouse lenses corresponding to a control group, a Prescription #2 group and a Prescription #3 group; and

FIG. 4 shows a result diagram of the effects of Prescription #4 to Prescription #6 on pupil diameter and pupil contraction time in various groups of presbyopic patients.

### DETAILED DESCRIPTION OF THE INVENTION

The present application is further elaborated below in conjunction with specific examples, which are only intended to explain the present application, rather than being construed as limitations to the present application. For a person of ordinary skill in the art, it will be understood that various changes, modifications, substitutions and variants can be made to these examples, without departing from the principle and spirit of the present application. The scope of the present application is defined by the appended claims and their equivalents. The experimental methods used in the following examples are conventional methods unless otherwise specified; and the reagents, materials, etc. used in the following examples may be obtained commercially unless otherwise specified.

Unless otherwise defined, all technical and scientific terms used herein have the same meaning as commonly understood by a person of ordinary skill in the art. In order to facilitate the understanding of the present application, the following terms involved in the present application are explained herein.

As used herein, the term “effective amount” refers to an amount that produces an effect on human and/or animals and is acceptable to human and/or animals. For example, a therapeutically or pharmaceutically effective amount is an amount of a drug required to produce a desired therapeutic effect, which may be reflected by the results of clinical trials, model animal studies, and/or in-vitro studies. The pharmaceutically effective amount depends on several factors, including (but not limited to): characteristic factors of a subject (e.g., the height, weight, sex, age, and drug history of the subject), and the severity of a disease. The active pharmaceutical ingredients (aceclidine, rebamipide and brimonidine) described in the present application together with pharmaceutically acceptable carriers and/or accessories (carriers and/or accessories for therapeutic administration, which are not essential active ingredients themselves and are not excessively toxic after administration) may constitute a pharmaceutical composition or pharmaceutical preparation. In a specific embodiment of the present application, the “effective amount” means an amount sufficient to inhibit, alleviate or prevent the development of presbyopia in a subject.

As used herein, the term “improve, alleviate or treat” refers to the medical management of patients with the aims of curing, improving, stabilizing or preventing a disease, pathological state or condition. This term includes an active therapy, that is, a treatment specifically aimed at improving a disease, pathological state or condition; and also includes an etiological treatment, that is, a treatment with the aim of removing the cause of a related disease, pathological state or condition. In addition, this term includes palliative care, that is, a treatment designed to alleviate symptoms rather than cure a disease, pathological state, or condition. This term also includes a prophylactic treatment, that is, a treatment aimed at minimizing or partially or completely suppressing the development of a related disease, pathological state or condition; and supportive care, i.e., a treatment that complements another specific therapy aimed at improving a related disease, pathological state or condition. In a specific embodiment of the present application, the term “improve, alleviate or treat” means the alleviation in the severity of symptoms of an ocular condition that adversely affects the visual acuity. Specifically, an ophthalmic preparation or a pharmaceutical composition described herein can be used to improve or treat presbyopic symptoms, such that a patient can visually focus on objects at a close range through the use of this preparation.

As used herein, the term “aceclidine” includes its salts, esters, analogues, prodrugs, and derivatives, including (but not limited to): aceclidine racemic mixtures, aceclidine (+) enantiomers, aceclidine (−) enantiomers, aceclidine analogues, and aceclidine prodrugs; the aceclidine analogues include (but are not limited to): 1,2,5 thiadiazole substituted analogues; and the aceclidine prodrugs include (but are not limited to) carbamate.

As used herein, the term “rebamipide”, including its salts, esters, analogues, prodrugs, and derivatives, including (but not limited to): rebamipide sodium salts, rebamipide choline salts, rebamipide tromethamine salts, rebamipide arginine salts, rebamipide lysine salts, and rebamipide magnesium salts.

As used herein, the term “brimonidine”, including its salts, esters, analogues, prodrugs, and derivatives, including (but not limited to): brimonidine tartrate, and 5-bromo-6-(2-imidazoline-2-ylamino)quinoxaline D-tartrate.

As used herein, the term “% (w/v)” refers to a weight percentage of a total composition.

As used herein, the term “about” refers to plus or minus 10% of all numerical values related to quantity, weight, etc. For example, “about 5% (w/v)” is understood to mean “4.5%-5.5% (w/v)”. Therefore, a value of up to 10% of the claimed quantity is included within the scope of the claim.

As used herein, the term “subject” is used to describe an animal, human or non-human treated with an ophthalmic preparation or pharmaceutical composition or method described in the present application. This disclosure anticipates human and veterinary applications. This term includes (but is not limited to): birds, reptiles, amphibians, and mammals such as human, other primates, pigs, rodents such as mice and rats, rabbits, guinea pigs, hamsters, horses, cattle, cats, dogs, sheep, chicken, and goats. In some embodiments, the subject is human, chicken, or mouse. In the preferred embodiment, the subject is human. Both pediatric and adult subjects are included. For example, the subjects described herein may be pediatric and adult subjects at least 6 months of age (e.g., 6 months or older, 12 months or older, 18 months or older, 2 years or older, 4 years or older, 6 years or older, 10 years or older, 13 years or older, 16 years or older, 18 years or older, 21 years or older, 25 years or older, 30 years or older, 35 years or older, 40 years or older, 45 years or older, 50 years or older, 60 years or older, 65 years or older, 70 years of age or older, 75 years of age or older, 80 years of age or older, 85 years of age or older, 90 years of age or older, or 2, 3, 4, 5, 6, 7, 8, 9, 10, 12, 13, 14, 15, 16, 18, 20, 21, 24, 25, 27, 28, 30, 33, 35, 37, 39, 40, 42, 44, 45, 48, 50, 52, 55, 60, 65, 70, 75, 80, 85, 90, 95, 96, 97, 98, 99, 100, 101, 102, 103, 104 years of age or older).

As used herein, the term “active ingredient”, refers to components of a pharmaceutically acceptable composition responsible for a therapeutic effect of the composition, while other components of the composition (e.g., excipients, carriers and diluents) are not responsible for the therapeutic effect of the composition, even if they have other functions (e.g., lubrication, flavoring, pH control, emulsification, stabilization, preservation, and other functions in addition to the therapeutic effect of the composition as described herein) in the composition that are necessary or desired as part of the preparation. Specifically, in some embodiments, the pharmaceutically acceptable composition described herein in which a compound of formula I is the only active ingredient with therapeutic activity is a composition in which no other component is present that will be considered to have therapeutic activity for the treatment or control of ocular conditions (e.g., presbyopia).

### Example 1 Drug Combination Screening Based on Target Information

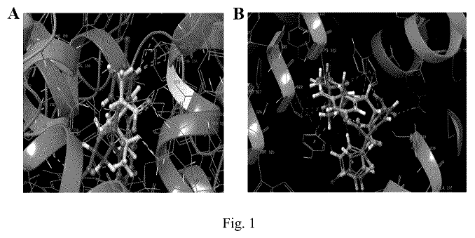

Based on the analysis of the previous literatures, it was found that the current drug mechanisms related to presbyopia were mainly as follows: 1) muscarinic acetylcholine receptor M3 agonists; 2) COX-2 inhibitors; and 3) antioxidant drugs. According to the key nodes, a pharmacological network model was recommended for drug combination screening. A COX-2 protein structure (PDB: 4PH9) and a muscarinic acetylcholine receptor (PDB: 4DAJ) were selected at the same time as key proteins. The results were combined with the pharmacological network model after virtual screening using a ZINC drug structure database, and scored on the basis of the weights of sides in a network. After screening, it was found that rebamipide ranked first among all drugs, and the first 20 molecules in the model included diclofenac (twelfth) and bromfenac (fourth), which were currently used in combination with drugs. In FIG. 1, Panel A shows screening in a combined mode of a screened drug with COX-2, and Panel B shows screening in a combined mode of a screened drug and a muscarinic acetylcholine receptor.

### Example 2 Method for Preparing Ophthalmic Preparation for the Treatment of Presbyopia

### 1. Method for Preparing Composition

### (1) Dissolution of Accessories

1) Dissolve carboxymethyl cellulose CMC (Macklin: C889437): a 2 L beaker was taken, about 1500 mL of ultrapure water at 80° C. was measured, carboxymethyl cellulose CMC was added slowly while stirring with an LED overhead stirrer, and other accessories were then added after stirring for about 7 h.

2) Add sodium chloride NaCl: a CMC solution was observed, and sodium chloride was added after the CMC was completely dissolved, and continued to stir for 10 min.

3) Add hydroxypropyl-β-cyclodextrin HPBCD (Bide: BD44359): hydroxypropyl-β-cyclodextrin HPBCD was added slowly and stirred for about 1 h until dissolved.

4) Add benzalkonium chloride BAK: benzalkonium chloride BAK was dissolved in about 50 mL of ultrapure water, then added dropwise to the CMC solution, and continued to stir for 10 min.

### (2) Adjustment of pH Value

The pH value was measured before the pH value was adjusted, and then a 1 mol/L NaOH solution or 1 mol/L HCl solution was added to adjust the pH value to about 6.9.

### (3) Filtration

An accessory solution was filtered with a 1.0/0.65 m filter cartridge.

### (4) Dissolution of Main Drug

1) A 100 mL beaker was taken, and 45 g of the accessory solution prepared above was measured.

2) Add active pharmaceutical ingredients in a prescription: the accessory solution was added with a prescribed amount of aceclidine (MedBio: MED17210) and brimonidine (α-2 adrenergic receptor agonist) (Bide: BD29454) while stirring to dissolve, then added with ultrafine crushed rebamipide particles (0.1-50 m), stirred magnetically for about 3 h, and then homogenize for 5 min.

### (5) Volume Metering

Each prescription was metered to a fixed weight of 50 g.

### (6) Filling

Multi-dose filling was performed with low-density polyethylene LDPE bottles, 5 mL/bottle. The concentrations of respective components in each prescription eye drops prepared were shown in Table 1.

TABLE 1
Concentrations of respective components of each prescription eye drops

| | #1 | #2 | #3 | #4 | #5 | #6 | #7 | #8 | #9 | #10 | #11 | #12 |
|---|---|---|---|---|---|---|---|---|---|---|---|---|
| Aceclidine (%, w/v) | 1.5 | 1.5 | 1.5 | 1.1 | 1.1 | 1.1 | 0.8 | 0.8 | 0.8 | | | |
| Pilocarpine (%, w/v) | | | | | | | | | | 1.25 | 1.25 | 1.25 |
| Rebamipide (%, w/v) | 0 | 1 | 2 | 0 | 1 | 2 | 0 | 1 | 2 | 0 | 1 | 2 |
| Brimonidine (%, w/v) | 0.02 | 0.02 | 0.02 | 0.02 | 0.02 | 0.02 | 0.02 | 0.02 | 0.02 | 0.02 | 0.02 | 0.02 |
| HPBCD (%, w/v) | 1.0 | 1.0 | 1.0 | 1.0 | 1.0 | 1.0 | 1.0 | 1.0 | 1.0 | 1.0 | 1.0 | 1.0 |
| NaCl (%, w/v) | 0.55 | 0.55 | 0.55 | 0.55 | 0.55 | 0.55 | 0.55 | 0.55 | 0.55 | 0.55 | 0.55 | 0.55 |
| CMC (%, w/v) | 0.05 | 0.05 | 0.05 | 0.05 | 0.05 | 0.05 | 0.05 | 0.05 | 0.05 | 0.05 | 0.05 | 0.05 |
| BAK (%, w/v) | 0.01 | 0.01 | 0.01 | 0.01 | 0.01 | 0.01 | 0.01 | 0.01 | 0.01 | 0.01 | 0.01 | 0.01 |
| NaOH (mmol) | q.s. | q.s. | q.s. | q.s. | q.s. | q.s. | q.s. | q.s. | q.s. | q.s. | q.s. | q.s. |

### 2. Experimental Results

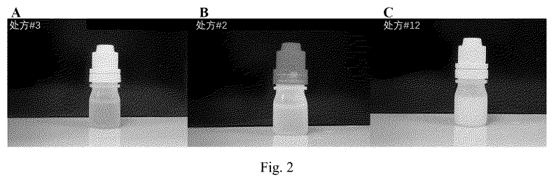

The results showed that Prescriptions #1 to #9 were all yellowy liquids, and Prescriptions #10 to #12 were milky suspensions with pH value of 6.9, osmotic pressure of 500 mOsm/kg, and viscosity of 4.2 cPa. The morphology diagrams of solutions corresponding to Prescriptions #3, #2 and #12 were shown in FIG. 2A-2C. Aceclidine and rebamipide could be combined to form a uniform and stable solution, while the solution formed by the combination of aceclidine, rebamipide and pilocarpine was an opaque, uneven and unstable suspension.

### Example 3 Animal Irritation Test

### 1. Experimental Method

Rabbits, which serve as research objects for an animal experiment, were randomly divided into a control group and experimental groups #1 to #9. For each experimental group, a single-dosage administration mode was used. The corresponding number of drug was added dropwise to the right eye, 50 μL each time. The control group was not administered with any drug. The left eye of the experimental group was administered with the same dosage of normal saline. The pupil size was examined by a slit lamp at 0 min, 10 min, 20 min, 30 min, 1 h, 2 h, 4 h, and 6 h after a single administration, and the degree of corneal opacity, conjunctival hyperemia, edema and secreta, iris hyperemia or hemorrhage were scored according to the Draize irritation test scoring criteria at 30 min, and the irritation was rated. The overall results showed that when the eye drops had an eye irritation score of less than 3, it could be considered that there was no obvious irritation, and the animal experiment had a good tolerance.

### 2. Experimental Results

The results showed that the eye irritation scores of Prescriptions #4 to #12 were all less than 3, that is, Prescriptions #4 to #12 had no significant irritation to the eyes of animals. Meanwhile, it was found that with the increase in the concentration of rebamipide, the duration of the aceclidine prescription at the same concentration increased, while pilocarpine had no similar synergistic effect with rebamipide. That is, there was no significant changes in the duration and irritation score in the pilocarpine prescription at the same concentration (see Table 2), indicating that there was a synergistic effect between rebamipide and aceclidine, while there was no synergistic effect between pilocarpine and rebamipide.

TABLE 2
Statistics of animal experiment results on each prescription eye drops

| | Prescription number | | | | |
|---|---|---|---|---|---|
| | #1 | #2 | #3 | #4 | #5 |
| | Pupil diameter (mm) | | | | |
| 0 min | 8.1 ± 0.2 | 8.2 ± 0.3 | 8.1 ± 0.3 | 8.3 ± 0.1 | 8.0 ± 0.2 |
| 10 min | 3.4 ± 0.2 | 3.2 ± 0.4 | 3.3 ± 0.3 | 3.1 ± 0.4 | 3.2 ± 0.3 |
| 20 min | 3.3 ± 0.3 | 3.3 ± 0.4 | 2.7 ± 0.2 | 3.1 ± 0.3 | 2.9 ± 0.5 |
| 30 min | 3.1 ± 0.6 | 3.1 ± 0.3 | 3.2 ± 0.4 | 3.3 ± 0.5 | 3.2 ± 0.6 |
| 1 h | 4.0 ± 0.5 | 4.1 ± 0.5 | 3.6 ± 0.5 | 4.2 ± 0.7 | 4.3 ± 0.4 |
| 2 h | 4.7 ± 0.6 | 5.1 ± 0.2 | 4.3 ± 0.6 | 5.4 ± 0.6 | 5.6 ± 0.6 |
| 4 h | 7.3 ± 0.5 | 7.2 ± 0.6 | 6.3 ± 0.7 | 7.4 ± 1.0 | 7.0 ± 0.7 |
| Irritation score | 4 | 4 | 3 | 2 | 2 |
| (15 min) | | | | | |
| | Prescription number | | | | |
| | #6 | #7 | #8 | #9 | #10 |
| | Pupil diameter (mm) | | | | |
| 0 min | 8.2 ± 0.4 | 8.3 ± 0.2 | 8.1 ± 0.1 | 8.2 ± 0.3 | 8.0 ± 0.2 |
| 10 min | 3.0 ± 0.6 | 3.5 ± 0.6 | 3.4 ± 0.6 | 3.4 ± 0.6 | 4.9 ± 0.6 |
| 20 min | 3.1 ± 0.6 | 3.3 ± 0.8 | 3.3 ± 0.4 | 3.4 ± 0.5 | 4.7 ± 0.5 |
| 30 min | 3.2 ± 0.4 | 3.4 ± 0.5 | 3.1 ± 0.5 | 3.5 ± 0.6 | 4.8 ± 0.6 |
| 1 h | 4.1 ± 0.5 | 4.2 ± 0.4 | 4.4 ± 0.4 | 4.2 ± 0.5 | 5.1 ± 0.5 |
| 2 h | 5.3 ± 0.6 | 5.2 ± 0.4 | 5.4 ± 0.5 | 5.1 ± 0.8 | 5.8 ± 0.8 |
| 4 h | 6.8 ± 0.7 | 7.6 ± 0.6 | 7.5 ± 0.4 | 7.2 ± 1.1 | 7.7 ± 0.9 |
| Irritation score | 1 | 0 | 0 | 0 | 2 |
| (15 min) | | | | | |
| | Prescription number | | | | |
| | | | Control | | |
| | #11 | #12 | group | | |
| | Pupil diameter (mm) | | | | |
| 0 min | 8.2 ± 0.3 | 8.1 ± 0.2 | 8.2 ± 0.4 | | |
| 10 min | 4.7 ± 0.6 | 4.8 ± 0.6 | 8.2 ± 0.3 | | |
| 20 min | 4.5 ± 0.5 | 4.4 ± 0.6 | 8.1 ± 0.3 | | |
| 30 min | 4.8 ± 0.5 | 4.9 ± 0.7 | 8.3 ± 0.4 | | |
| 1 h | 5.2 ± 0.4 | 5.3 ± 0.5 | 8.1 ± 0.4 | | |
| 2 h | 5.9 ± 0.7 | 5.8 ± 0.8 | 8.2 ± 0.4 | | |
| 4 h | 7.9 ± 0.6 | 7.5 ± 0.9 | 8.1 ± 0.4 | | |
| Irritation score | 2 | 2 | 0 | | |
| (15 min) | | | | | |

### Example 4 Effects of Rebamipide Eye Drops on Lens Sclerosis in C57BL/6 Mice

### 1. Experimental Method

10-week-old male C57BL/6 mice were divided into three groups (n=3 mice/group), administrated by eye drop with water, Prescription #2, and Prescription #3 for 28 weeks, once a day consecutively for 24 weeks. The above mice administrated by eye drop with water, Prescription #2, and Prescription #3 were a control group, a Prescription #2 group, a Prescription #3 group, respectively. The lens elasticity of the mice in each group was measured using Softmeasure HG1003-SL. After euthanasia of each mouse, the lens was removed intermediately and placed on a scaffold located on the posterior pole parallel to the bottom. A tip of an altimeter was turned down, so that a pressure was applied to the lens, and then the degree of lens strain was measured. The lens elasticity was calculated using the degrees of pressure and strain on the lens. A t-test was used for comparison between two groups, and P<0.05 was a significant difference.

### 2. Experimental Results

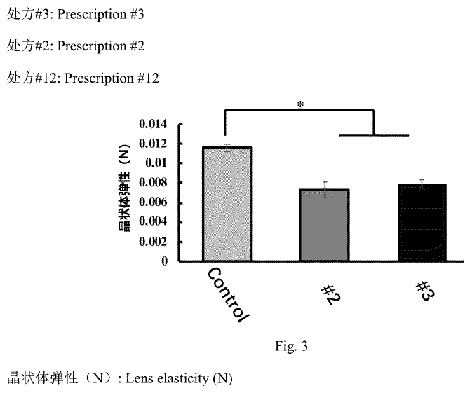

The results showed the lens elasticity had a significant improvement in the mice administrated by eye drop with rebamipide (Prescription #2, Prescription #3) for 28 weeks relative to the control group (see FIG. 3).

### Example 5 Effects of Different Prescription Compositions Prepared in the Present Application on Presbyopic Patients

### 1. Experimental Method

A total of 18 patients aged 50-60 years with presbyopia above 2.0 D were randomly divided into three groups. After full notification and signing of an experimental informed consent form, Prescription #4-6 eye drops with no obvious irritating effects were selected for single-eye (OD) drop, and a series of eye examinations were performed. The eye examinations include: 1) distant vision of naked eyes: measured using a Snellen chart; 2) near vision of naked eyes: tested using a handheld vision chart; 3) pupil diameter: measured at baseline time, 1 h, 4 h, and 6 h after drugs were added dropwise.

### 2. Experimental Results

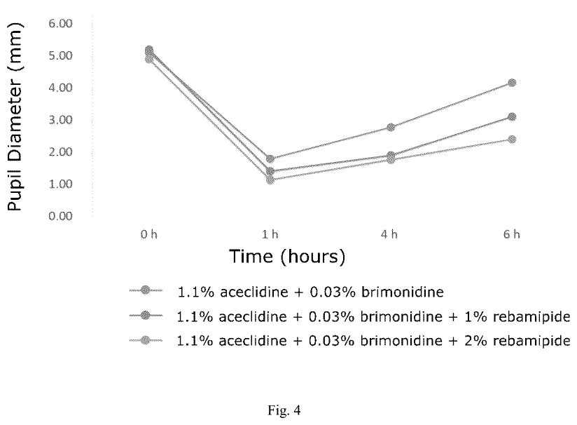

The results showed that the mean pupil diameter at baseline was 5.1±0.4 mm, and the pupil diameter was reduced to 2.1±0.4 mm after 1 h of drop with each prescription, and had no significant difference among the respective groups one hour later; the pupil diameter of the same group was significantly reduced after 1 h of eye drop compared with baseline; there were no side effects such as eye redness, tingling, supraorbital neuralgia and tingling among the groups; the starting time of pupil contraction was 15±3.0 min, the near vision at baseline was 8 pt in each group, and 4 pt after 1 h; the durations (defined as the time to return vision before medication) were as follows: 8.4±1.1 h for Prescription #4; 9.3±1.5 h for Prescription #5; 10.5±1.3 h for Prescription #6; and the pupil diameters at different time points in each group were shown in FIG. 4. In the study, the inventors found that there was a dosage effect between the concentration of rebamipide and the time of pupil contraction, and there were also no side effects such as eye redness, tingling, supraorbital neuralgia and tingling in all three prescriptions.

The description of the above examples is only for understanding the method of the present application and its core idea. It should be pointed out that a person of ordinary skill in the art may also make several improvements and modifications on the present application without departing from the principles of the present application, which should also fall within the protection scope of the present application.

1. An ophthalmic preparation for improving, alleviating or treating presbyopia, containing effective amounts of muscarinic acetylcholine receptor M3 agonist and rebamipide.

2. The ophthalmic preparation according to claim 1, wherein the muscarinic acetylcholine receptor M3 agonist comprises aceclidine, pilocarpine, carbachol, methacholine, cevimeline, talsaclidine, acetylcholine, huperzine A, milameline, DREADD agonist 21, alvameline, carbamylmethylcholine, sabcomeline, and/or arecoline.

3. The ophthalmic preparation according to claim 2, wherein the muscarinic acetylcholine receptor M3 agonist is aceclidine.

4. The ophthalmic preparation according to claim 2, further comprising an α-2 adrenergic receptor agonist.

5. The ophthalmic preparation according to claim 4, wherein the α-2 adrenergic receptor agonist is brimonidine, medetomidine, guanfacine, clonidine, dexmedetomidine, apraclonidine, mivazerol, naphazoline, oxymetazoline, tetrahydrozoline, xylazine, tizanidine, methylnorepinephrine, moxonidine, and/or rilmenidine.

6. The ophthalmic preparation according to claim 5, wherein the α-2 adrenergic receptor agonist is brimonidine.

7. The ophthalmic preparation according to claim 5, further comprising a viscosity enhancer.

8. The ophthalmic preparation according to claim 7, wherein the viscosity enhancer is carboxymethyl cellulose, methylcellulose, hydroxypropyl methylcellulose, polyethylene glycol, povidone, glycerin, polyvinyl alcohol, polyvinylpyrrolidone, polyalkyl styrene, polymethacrylate, and/or polyacrylate.

9. The ophthalmic preparation according to claim 8, wherein the viscosity enhancer is carboxymethyl cellulose.

10. The ophthalmic preparation according to claim 8, further comprising a surfactant.

11. The ophthalmic preparation according to claim 10, wherein the surfactant is hydroxypropyl-β-cyclodextrin, polyoxyethylene alkyl ether, Tween, sodium lauryl sulfate, sodium laureth sulfate, poloxamer, polysorbate, sorbitan monopalmitate, sorbitan monostearate, sorbitan monooleate, and/or polyethylene glycol alkyl.

12. The ophthalmic preparation according to claim 10, wherein the surfactant is hydroxypropyl-β-cyclodextrin.

13. The ophthalmic preparation according to claim 1, wherein the rebamipide has a concentration of 0.50%-4.00% (w/v).

14. The ophthalmic preparation according to claim 3, wherein the aceclidine has a concentration of 0.50%-1.50% (w/v).

15. The ophthalmic preparation according to claim 6, wherein the brimonidine has a concentration of 0.01%-1.00% (w/v).

16. The ophthalmic preparation according to claim 9, wherein the carboxymethyl cellulose has a concentration of 0.03%-0.50% (w/v).

17. The ophthalmic preparation according to claim 12, wherein the hydroxypropyl-β-cyclodextrin has a concentration of 0.01%-5.00% (w/v).

18. A method for preparing the ophthalmic preparation according to claim 1, comprising: combining effective amounts of muscarinic acetylcholine receptor M3 agonist and rebamipide with a pharmaceutically acceptable carrier and/or accessory.

19. A method for improving, alleviating or treating presbyopia, mild hyperopia, irregular astigmatism and/or hyperopic accommodative esotropia, or increasing the visual depth of field, or contracting pupils, or alleviating the progression of a long-term or medium-term course of presbyopia, comprising: applying an effective amount of the ophthalmic preparation according to claim 1 to affected eyes of a subject in need.

20. The method according to claim 19, wherein the muscarinic acetylcholine receptor M3 agonist is aceclidine, pilocarpine, carbachol, methacholine, cevimeline, talsaclidine, acetylcholine, huperzine A, milameline, DREADD agonist 21, alvameline, carbamylmethylcholine, sabcomeline, and/or arecoline.
